(19) United States
(12) Patent Application Publication
(10) Pub. No.: US 2024/0307211 A1
(43) Pub. Date: Sep. 19, 2024

(54) SEALING DEVICE FOR OSTOMY BAGS

(71) Applicant: Brett L. Dunbar, Philadelphia, PA (US)

(72) Inventor: Brett L. Dunbar, Philadelphia, PA (US)

(21) Appl. No.: 18/121,129

(22) Filed: Mar. 14, 2023

(51) Int. Cl. A61F 5/44; A61F 5/445

(52) U.S. Cl. CPC A61F 5/4407; A61F 5/445

(57) ABSTRACT

A sealing device for an ostomy bag is shown and described. The sealing device for an ostomy bag includes a first material having a top surface and a bottom surface, wherein the material is malleable. The first material has an adhesive along the bottom surface. The adhesive will adhere to skin. The first material can for formed into various shapes having a perimeter formed by the first material and an opening therebetween.

### BACKGROUND OF THE INVENTION

The present invention relates to Stoma and Ostomy bag connections. More particularly, the present invention provides a device which will allow for a better seal between a user's skin and an ostomy bag.

The present ostomy bag system-while necessary for various procedures and medical functions, leaves much to be desired. Inventors are constantly attempting to improve the fit, function, and ease of use for patients required to use ostomy bags. However, while each innovation improves the use of the ostomy bag, nothing currently exists to solve the issues created by ostomy bags.

One issue that is created by the nature of an ostomy bag is the smell of the contents. While this issue is not formally addressed by this invention, it is worth mentioning here. If the materials departed from a user stay within an ostomy bag, then the smell is not an issue. However, one of the major issues of an ostomy bag is leaking. Often times, ostomy bags leak due to poor seals. These seals can be weakened by any number of accidents or unfortunate events. Some of these items include: the bag contents resting around the seal of the bag, the bag being pulled away from the user, and the contents not making it fully into the bag.

Attempts have been made to resolve these issues. Often times, by adding a pad around the ostomy opening. However, these pads do not usually accomplish the mission. Often these pads keep the leakage away from the skin, but they do not prevent the leak. The present solutions do no seek to eliminate leaks but to limit the effects thereof.

Consequently, there is a need for an improvement in the art of sealing an ostomy bag to a user. The present invention substantially diverges in design elements from the known art while at the same time solves a problem many people face when using ostomy bags. In this regard the present invention substantially fulfills these needs.

### SUMMARY OF THE INVENTION

The present invention provides a sealing device for an ostomy bag. wherein the same can be utilized for providing comfort and peace of mind to a user when using an ostomy bag. The sealing device for an ostomy bag includes a first material having a top surface and a bottom surface, wherein the material is malleable. The first material has an adhesive along the bottom surface. The adhesive will adhere to skin. The first material can for formed into various shapes having a perimeter formed by the first material and an opening therebetween.

Another object of the sealing device for an ostomy bag is to have the first material be pre-formed into specific shapes.

Another object of the sealing device for an ostomy bag is to have the first material be pre-formed into specific shapes of specific sizes.

Another object of the sealing device for an ostomy bag is to have a thin sheet of material removably attached to the adhesive.

Another object of the sealing device for an ostomy bag is to have the first material have a thickness of at least one quarter of an inch.

Another object of the sealing device for an ostomy bag is to have the sheet of first material able to be formed into various shapes having a perimeter formed by the material and an opening therebetween by separating the sheet of first material.

Another object of the sealing device for an ostomy bag is to have the sheet of first material be perforated such that pieces of a specific shape can be separated from the sheet of first material.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of a sealing device for an ostomy bag.

FIG. 2 shows a second perspective view of an embodiment of the sealing device for an ostomy bag.

FIG. 3 shows a perspective in-use view of an embodiment of the sealing device for an ostomy bag.

FIG. 4 shows a second perspective in-use view of an embodiment of the sealing device for an ostomy bag.

FIG. 5 shows a cross-sectional in-use view of an embodiment of the sealing device for an ostomy bag.

### LIST OF REFERENCE NUMERALS

With regard to the reference numerals used, the following numbering is used throughout the drawings.

| | |
|---|---|
| 100 | Sealing Device For Ostomy Bag |
| 101 | Sheet of Material |
| 102 | Adhesive Layer |
| 103 | Removable Cover |
| 201 | Aperture |
| 301 | Pieces of Material |
| 302 | Stoma |
| 401 | Device |
| 501 | Ostomy Bag |

### DETAILED DESCRIPTION OF THE INVENTION

Reference is made herein to the attached drawings. Like reference numerals are used throughout the drawings to depict like or similar elements of the sealing device for an ostomy bag. For the purposes of presenting a brief and clear description of the present invention, a preferred embodiment will be discussed as used for the sealing device for an ostomy bag. The figures are intended for representative purposes only and should not be considered to be limiting in any respect.

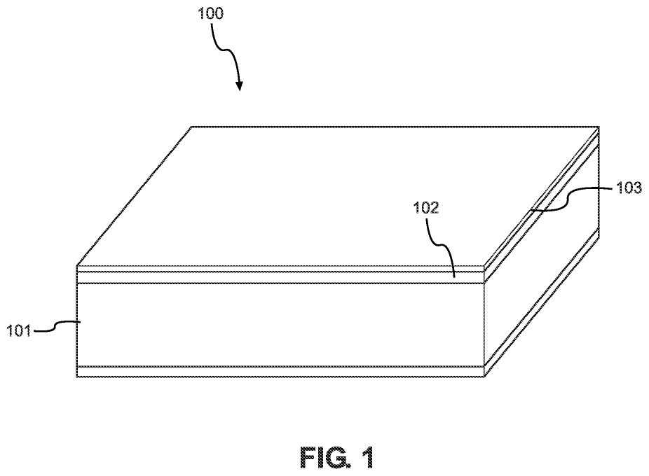

Referring now to FIG. 1, there is shown a perspective view of an embodiment of a sealing device for an ostomy bag. In one embodiment the sealing device for an ostomy bag 100 is comprised of a flexible sheet of material 101. In one embodiment the sheet of material 101 is made from a synthetic rubber. Specifically, in one embodiment the sheet of material 101 is made from the synthetic rubber SBR. In a further embodiment the sheet of material 101 may be further mixed with a blended latex foam. This configuration meets the FDA regulations for external use on the body as of the filing date.

In one embodiment the sheet of material 101 has an adhesive layer 102 on one side. This adhesive layer 102 is capable of adhering to skin. In one embodiment the adhesive layer 102 has a removable cover 103 placed thereon. This will ensure that the adhesive 102 stays fresh and does not prematurely adhere to the wrong surface. In a further embodiment the adhesive for the adhesive layer 102 is made from FDA approved adhesive for application to the skin. In a further embodiment, the adhesive is one that has been medically proven not to irritate the skin.

In the shown embodiment each layer, the sheet of material 10, any included adhesive layers and associated covers will be able to be cut. This will allow for the user to cut the sheet of material 101 into necessary sizes and shaped. In another embodiment the sheet of first material 101 and the included adhesive layers and associated covers will be perforated. This will allow a user to remove a pre sized piece from the sheet of material 101.

Part of what allows the sealing device for an ostomy bag 100 to function is the thickness of the sheet of material 101. In the currently solutions of the prior art the devices for ostomy bags are thin and designed more for absorption than leak prevention itself. Here the sealing device for an ostomy bag 100 is designed to prevent the leak. This is accomplished by having a minimal thickness. In one embodiment the sheet of material 101 is no more than a quarter inch thick. In another embodiment three eighths of an inch is used. In another embodiment the sheet of material 101 has a thickness of at least a half an inch. Greater thicknesses may be used to create the desired effects as described below. Different ostomy bags and connections will require different thickness as one of ordinary skill in the art will understand.

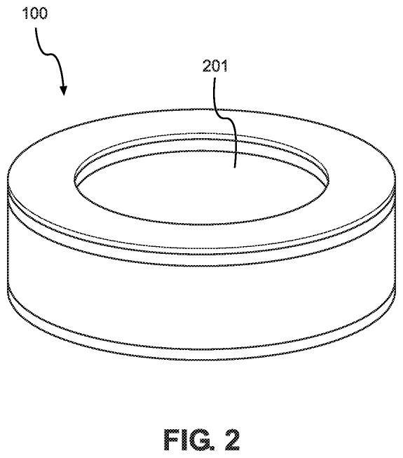

Referring now to FIG. 2, there is shown a second perspective view of an embodiment of the sealing device for an ostomy bag. In some embodiments the sheet of material 101 described in FIG. 1 is preformed into desired shapes and sizes. In one embodiment the sealing device for an ostomy bag 100 is formed into a circle having an aperture 201 locating within the center of the device. In another embodiment the sealing device for an ostomy bag 100 is formed into a rectangle having an aperture 201 locating within the center of the device. These precut devices will allow a user to simply take a device and apply it around the stoma.

In different embodiments the preformed sealing device for an ostomy bag 100 may come in various shapes and sizes. For example, in on embodiment the corners of the sealing device for an ostomy bag 100 may be rounded. This will help to eliminate pressure applied to an ostomy bag. In another embodiment the sealing device for an ostomy bag 100 may be an oval.

In different embodiments different sized sealing devices for an ostomy bags 100 will accommodate different types and sizes of ostomy bags. It should be noted that one of ordinary skill in the art will be able to determine the best size for each bag type. The size of the sealing device for an ostomy bag 100 does not have to be a 1 to 1 comparison. Meaning that one size of sealing device for an ostomy bag 100 may work with several different ostomy bags.

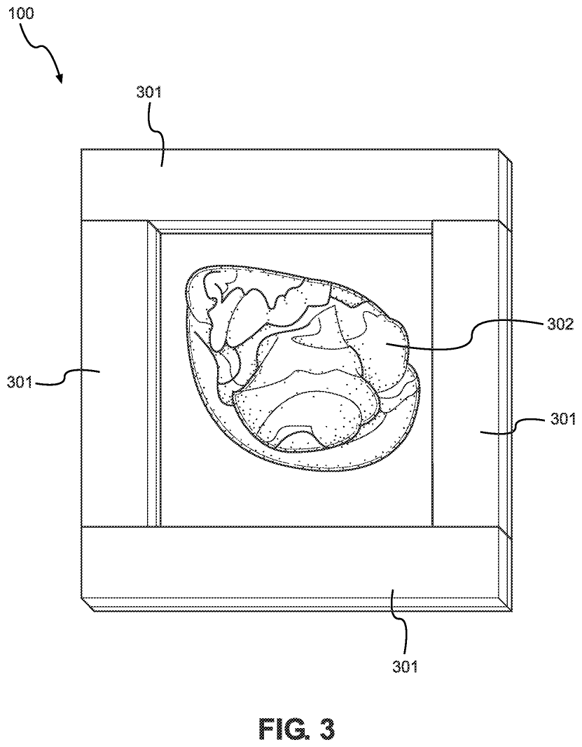

Referring now to FIG. 3, there is shown a perspective in-use view of an embodiment of the sealing device for an ostomy bag. In FIG. 3 there is shown a custom shaped sealing device for an ostomy bag 100. In this embodiment the user began with the sheet of material 101 described in FIG. 1. The sheet of material 101 is then cut into pieces 301 of the desired size. The pieces 301 may then be attached to either the user's body around a stoma 302 or around the hole of ostomy bag. In the shown embodiment the pieces 301 are placed around the stoma 302. After the pieces 301 are set in place an ostomy bag can then be attached therearound. In the embodiment where the sheet of material 101 has a second layer of adhesive the adhesive can be used to stick the blocks to the ostomy bag.

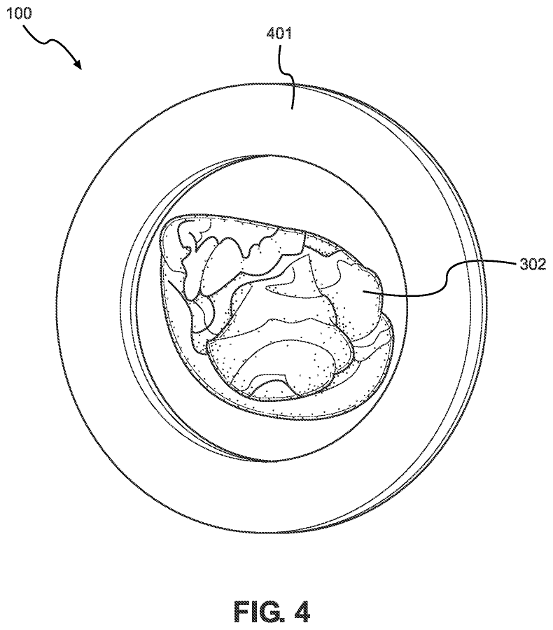

Referring now to FIG. 4, there is shown a second perspective in-use view of an embodiment of the sealing device for an ostomy bag. In FIG. 4 there is shown a pre-shaped sealing device for an ostomy bag 100. In this embodiment the user began with the device 401 as described in FIG. 2. The user may then attach the device 401 to either the user's body around a stoma 302 or around the hole of ostomy bag. In the shown embodiment the device 401 is placed around the stoma 302. After the device 401 is set in place an ostomy bag can then be attached therearound. In the embodiment where the device has a second layer of adhesive the adhesive can be used to stick the blocks to the ostomy bag.

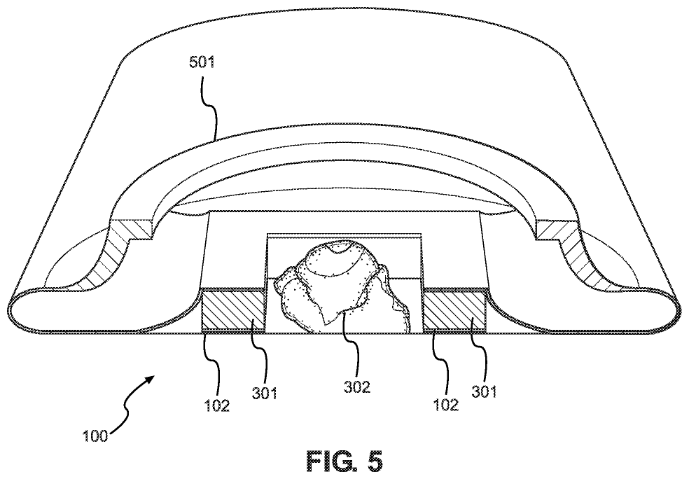

Referring now to FIG. 5, there is shown a cross-sectional in-use view of an embodiment of the sealing device for an ostomy bag. This view shows use of the sheet of material cut 101 into pieces 301 and placed to a desired shape and size. The pieces 301 are shown placed round the stoma 302. The pieces are held in place via the adhesive layer 102. In this embodiment the ostomy bag 501 is then secured to the user within the middle opening created by the pieces 301. The side of the device 401 pointing away from the user is then adhesively secured to the ostomy bag 501. This adds extra adhesive between the user and the bag. This means that the bag is less likely to separate from the user.

In this configuration the device creates a raised section around the stoma 302. Fluids from the stoma 302 will be able to enter the ostomy bag 501 and gravity will pull them away from the stoma 302. The raise section created by the pieces 301 will prevent the same fluids from re-entering the portion of the bag near the stoma 302. This will keep fluids away from the adhesive portions of the connection and will help to prevent leaks in the ostomy bag 501.

By using the sealing device for an ostomy bag, leaks are less likely to occur during use of an ostomy bag 501. Further, the life of a bag may be increased by several days. This means that a wearer will not have to remove bags as often, which can help to reduce discomfort. This can also help to reduce the amount of supplies needed. According to the industry standard, an ostomy bag is designed to last

three days. This is also the standard insurance companies use when covering ostomy bags. This means that if a bag becomes in operative after only one day a user is one their own for the bag to cover the other two days. Using this device can extend the life of an ostomy bag to five days. This means that a user will on average need less than ten bags a month. This will ensure that a user's insurance will cover not only the minimum amount of bags required but extra. This can significantly reduce stress in a user's life.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A sealing device for an ostomy bag, the device comprising:
A first material having a top surface and a bottom surface, wherein the material is malleable;
The first material has an adhesive along the bottom surface, wherein the adhesive will adhere to skin;
Wherein the first material can for formed into various shapes having a perimeter formed by the first material and an opening therebetween.

2. The sealing device for an ostomy bag of claim 1, wherein the first material is pre-formed into specific shapes.

3. The sealing device for an ostomy bag of claim 1, wherein the first material is pre-formed into specific shapes of specific sizes.

4. The sealing device for an ostomy bag of claim 1, further comprising a thin sheet of material removably attached to the adhesive.

5. The sealing device for an ostomy bag of claim 1, wherein the first material has a thickness of at least one quarter of an inch.

6. A sealing device for an ostomy bag, the device comprising:
A sheet first material having a top surface and a bottom surface, wherein the material is malleable;
The first material has an adhesive along bottom surface, wherein the adhesive will adhere to skin;
Wherein the sheet of first material can for formed into various shapes having a perimeter formed by the material and an opening therebetween by separating the sheet of first material.

7. The sealing device for an ostomy bag of claim 1, wherein the sheet of first material is perforated such that pieces of a specific shape can be separated from the sheet of first material.

8. The sealing device for an ostomy bag of claim 1, further comprising a thin sheet of material removably attached to the adhesive.
The sealing device for an ostomy bag of claim 1, wherein the first material has a thickness of at least one quarter of an inch.
